(19) United States
(12) Patent Application Publication
(10) Pub. No.: US 2026/0007667 A1
(43) Pub. Date: Jan. 8, 2026

(54) TOPICAL ECTODERMAL STIMULATION

(71) Applicant: SOLIFON PHARMACEUTICALS, INC., Dallas, TX (US)

(72) Inventors: Ezekiel Fink, Dallas, TX (US); Ranjeet Patil, Houston, TX (US)

(73) Assignee: SOLIFON PHARMACEUTICALS, INC., Dallas, TX (US)

(21) Appl. No.: 19/131,618

(22) PCT Filed: Nov. 22, 2023
(86) PCT No.: PCT/US2023/080960

§ 371 Date: May 21, 2025

Related U.S. Application Data
(60) Provisional application No. 63/384,638, filed on Nov. 22, 2022.
(60) Provisional application No. 63/601,088, filed on Nov. 20, 2023.

(51) Int. Cl. A61K 31/506; A61K 9/00; A61K 47/12

(52) U.S. Cl. CPC A61K 31/506; A61K 9/0014; A61K 47/12

(57) ABSTRACT

Apparatus and associated methods relate to inducing ectodermal stimulation by substantially continuous topical application of a therapeutic dose of vascular growth induction agent (VGIA) and moisturizing compound. The moisturizing compound may, for example promote skin health and/or penetration of the VGIA to target cells. The VGIA may, for example, promote vascularization, neurostimulation, and/or oxygenation. In an illustrative example, the VGIA may, for example, include minoxidil. The moisturizing compound may, for example, include lactic acid. In some implementations, the VGIA and moisturizing compound may be substantially continuously topically applied by a cream. In some implementations, the VGIA and moisturizing compound may be substantially continuously topically applied by a controlled-release application device. Various embodiments may advantageously induce reversal of disease processes at the ectodermal level in affected peripheral body portions (e.g., feet, legs, hands).

### CROSS-REFERENCE TO RELATED APPLICATIONS

This application claims the benefit of U.S. Provisional Application Ser. No. 63/384,638, titled “Topical Ectodermal Stimulation,” filed by Ezekiel Fink on Nov. 22, 2022. This application also claims the benefit of U.S. Provisional Application Ser. No. 63/601,088, titled “Articles Evidencing Long Felt Unmet Need,” filed by Ezekiel Fink on Nov. 20, 2023. This application incorporates the entire contents of the foregoing applications herein by reference.

The subject matter of this application may have common inventorship with and/or may be related to the subject matter of U.S. application Ser. No. 12/070,930, titled “Topical Treatment of Peripheral Diabetic Complications,” filed Feb. 23, 2008 by Ezekiel Fink, and published Jan. 15, 2009 as U.S. Patent Application Publication US 2009/0018151. This application incorporates the entire contents of the foregoing application herein by reference.

### TECHNICAL FIELD

Various embodiments relate generally to topical ectodermal stimulation.

### BACKGROUND

Patients may suffer from pathologies that impact peripheral body portions. Such pathologies may, for example, include neuropathies. In some examples, neuropathies impacting peripheral body portions may include diabetic neuropathy. Diabetic neuropathy may, for example, cause loss of hair, loss of feeling, and/or skin.

### SUMMARY

Apparatus and associated methods relate to inducing ectodermal stimulation by substantially continuous topical application of a therapeutic dose of vascular growth induction agent (VGIA) and moisturizing compound. The moisturizing compound may, for example promote skin health and/or penetration of the VGIA to target cells. The VGIA may, for example, promote vascularization, neurostimulation, and/or oxygenation. In an illustrative example, the VGIA may, for example, include minoxidil. The moisturizing compound may, for example, include lactic acid. In some implementations, the VGIA and moisturizing compound may be substantially continuously topically applied by a cream. In some implementations, the VGIA and moisturizing compound may be substantially continuously topically applied by a controlled-release application device. Various embodiments may advantageously induce reversal of disease processes at the ectodermal level in affected peripheral body portions (e.g., feet, legs, hands).

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an illustrative progression of topical ectodermal stimulation using a topical ectodermal stimulation compound (TESC) employed in an illustrative use-case scenario.

FIG. 2A depicts an exemplary chemical structure of minoxidil.

FIG. 2B depicts an exemplary chemical structure of lactic acid.

FIG. 3 is a flowchart illustrating an exemplary topical ectodermal stimulation method.

FIG. 4 depicts an exemplary field-activated continuous topical ectodermal delivery device (CTEDD).

FIG. 5 depicts activation of a field-activated CTEDD.

FIG. 6 depicts illustrative active ingredient concentration profiles in a topical ectodermal stimulation method, such as with and without a CTEDD.

FIG. 7 depicts an illustrative kit and illustrative replaceable topical ectodermal delivery devices (RTEDDs).

FIG. 8 depicts a block diagram of an illustrative automatic topical ectodermal delivery device (ATEDD).

FIG. 9 depicts an exemplary adhesive CTEDD for a foot in an illustrative use-case scenario.

FIG. 10 depicts exemplary adhesive CTEDDs for a foot.

FIG. 11, FIG. 12, FIG. 13, and FIG. 14 depict the exemplary adhesive CTEDDs of FIG. 10 in illustrative use-case scenarios.

FIG. 15 depicts an illustrative portion of a CTEDD having spatially distributed TESC.

FIG. 16 depicts an illustrative TESC having active ingredient carriers configured to selectively release active ingredient.

Like reference symbols in the various drawings indicate like elements.

### DETAILED DESCRIPTION OF ILLUSTRATIVE EMBODIMENTS

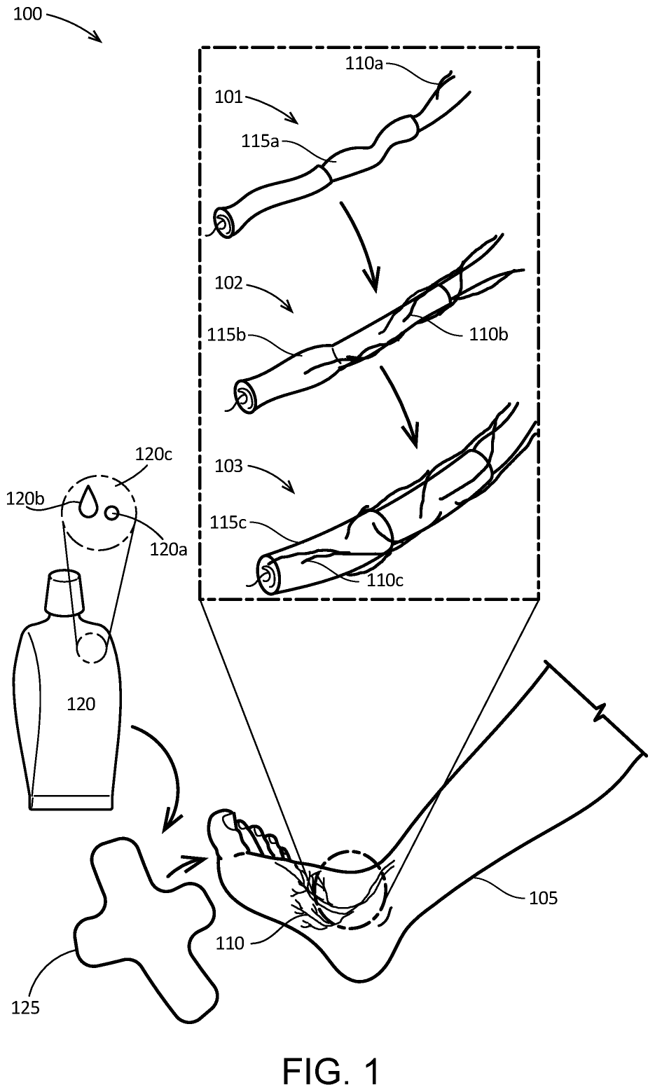

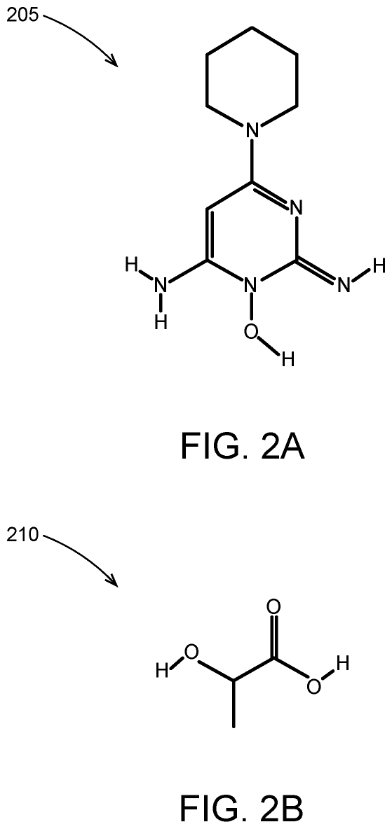

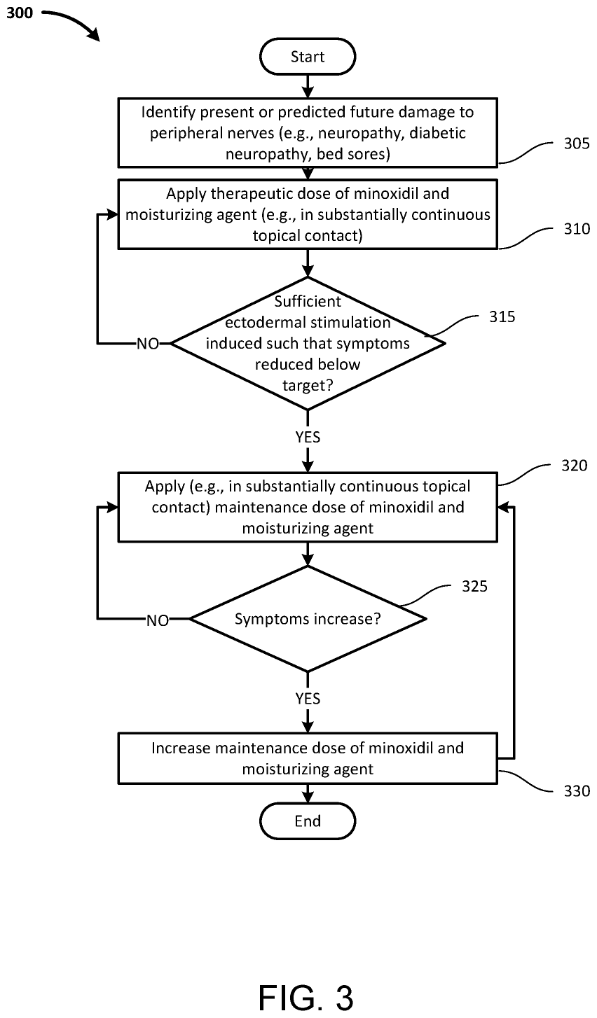

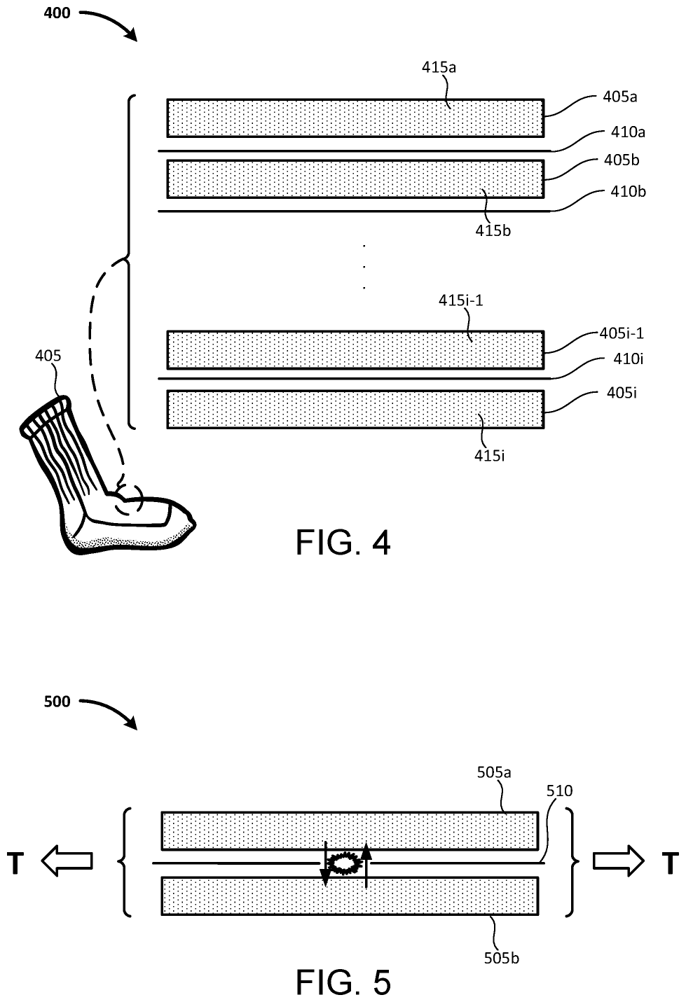

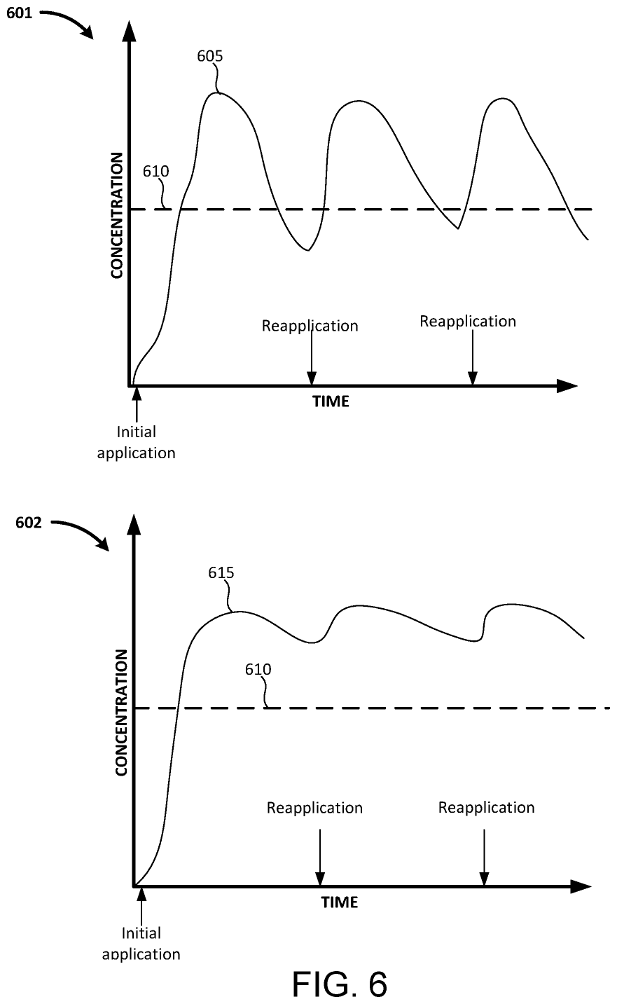

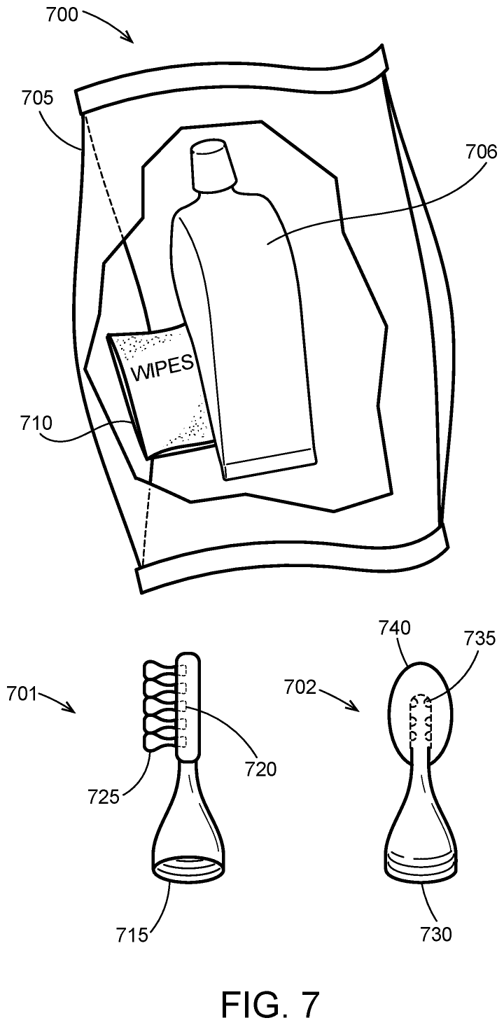

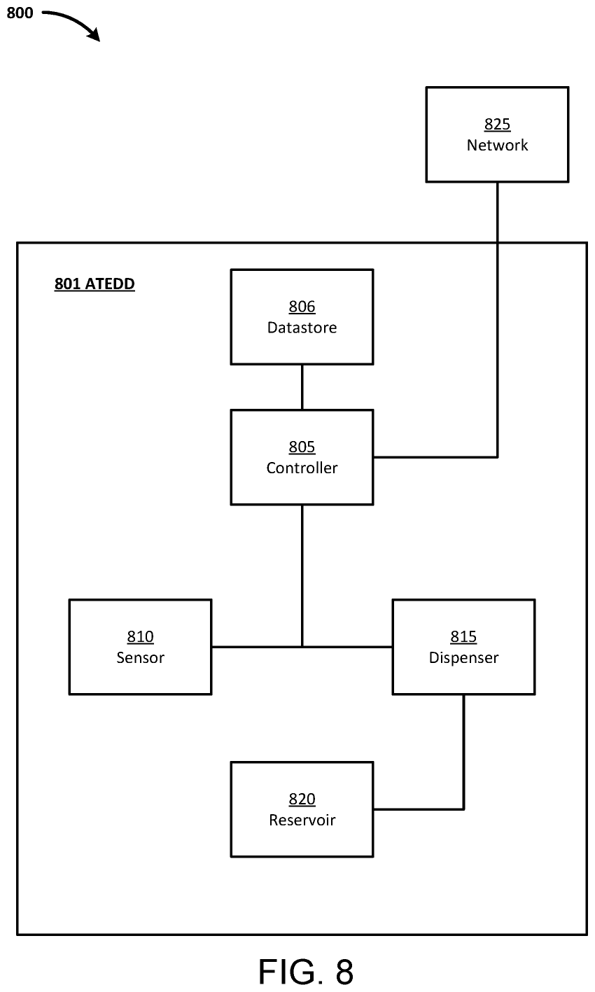

To aid understanding, this document is organized as follows. First, to help introduce discussion of various embodiments, a topical ectodermal stimulation composition (TESC) and associated method is introduced with reference to FIGS. 1-2B. Second, that introduction leads into a description with reference to FIG. 3 of an exemplary TESC therapy method. Third, with reference to FIGS. 4-5, a continuous topical ectodermal delivery device (CTEDD) is described. Fourth, with reference to FIG. 6, the discussion turns to exemplary advantages. Sixth, this disclosure turns to a review of experimental data relating to topical ectodermal stimulation using a particular TESC. Finally, the document discusses further embodiments, exemplary applications and aspects relating to TESC and associated methods and apparatus.

The ectoderm, which is an embryonic tissue layer, gives rise to the skin, the brain, the spinal cord, peripheral nerves, hair, nails, and/or sweat glands. Diseases which impact one element of the ectoderm may, for example, impact other components of the ectoderm. As an illustrative example, diabetes may impact the peripheral nervous system (PNS). A common complication of diabetes on the PNS may, for example, include distal symmetric peripheral neuropathy (DSPN). In some examples, DSPN may be initially expressed in the feet first. DSPN may, for example, then migrate centrally. Hair follicles in diabetics may, for example, be lost in a similar distribution as DSPN. Skin dryness may, for example, also occur in a similar distribution as in DSPN.

FIG. 1 depicts an illustrative progression of topical ectodermal stimulation using a topical ectodermal stimulation (TESC) employed in an illustrative use-case scenario. As shown in an illustrative stimulation progression use-case scenario 100, a peripheral body portion 105 (e.g., a limb, a leg as shown) may be afflicted by a pathology. The pathology may, by way of example and not limitation, affect one or more dermal layers (e.g., skin), nails, and/or hair. As an illustrative example, the pathology may include a neuropathy. The neuropathy may, for example, be a diabetic neuropathy.

For example, degradation of the vasculature 110 may cause degradation of ectodermal tissue (e.g., including short fiber nerves). Ectodermal tissue may, by way of example and not limitation, include skin (e.g., dermal tissue). Ectodermal tissue may, for example, include nerves. Ectodermal tissue may, for example, include hair. Ectodermal tissue may, for example, include nails.

As shown in exemplary scenario 101, degradation of the peripheral vasculature 110a results in degradation of nerve 115a. Accordingly, a patient may, by way of example and not limitation, lose sensation (e.g., feeling) in their peripheral body portion 105. In some examples, the patient may suffer hair loss due to loss of peripheral vasculature. The patient may, for example, suffer loss of nails (e.g., toenails, fingernails) from loss of peripheral vasculature. In some examples, the patient may suffer dermal damage (e.g., sluffing, dryness, thinning, sores, necrosis) due to loss of peripheral vasculature.

A TESC 120 may, for example, be applied to the skin. The TESC 120 may, for example, include a vasculature growth induction agent (VGIA 120a). The TESC 120 may, for example, include a moisturizing agent 120b. The TESC 120 may, for example, be in a base 120c configured to induce extended adherence of the TESC 120 to the external surface of the peripheral body portion 105. Extended adherence may, for example, maintain extended physical contact of the moisturizing agent 120b and/or the VGIA 120a with one or more portions of the ectoderm. The moisturizing agent 120b may, for example, soften the skin (e.g., the epidermis) and promote transport of the VGIA 120a into and/or through the skin.

In some implementations, such as depicted, the TESC 120 may be applied to the peripheral body portion 105 via an applicator. For example, the applicator may include a bandage 125, such as shown. For example, the applicator may advantageously promote even distribution of the TESC 120 across the peripheral body portion 105. For example, the bandage 125 may advantageously promote prolonged contact and/or sustained release of the TESC 120.

Extended physical contact of the vasculature 110 with the VGIA 120a, for example, may promote and/or induce regenerative growth of the VGIA 120a. The VGIA 120a may, for example, induce budding and development of peripheral vasculature (e.g., capillaries), as shown in exemplary scenario 102. As shown in exemplary scenario 102, the vasculature 110b is growing and expanding. Increased vascularization may induce reversal of the disease process. For example, the vasculature 110b extending further along the nerve 115b may induce regeneration and/or growth of the nerve 115b. For example, the vasculature 110b may improve oxygenation, nutrient availability, and/or waste removal along the nerve 115b. Restoration of the vasculature 110b and/or the nerve 115b may, for example, induce and/or promote restoration of sense (e.g., ‘feeling’) and/or ectodermal growth (e.g., hair, nails, skin, nerves).

Continued physical contact of the peripheral vasculature 110a and/or the vasculature 110b with the epidermis may, for example, continue promoting and/or inducing regeneration and/or growth (e.g., new) of the vasculature 110, as shown in exemplary scenario 103. The continued regeneration and/or extension of the peripheral vasculature 110 may, for example, induce and/or promote continued reversal of the disease process. For example, the vasculature 110c may extend to perfuse the entire nerve 115c, inducing regeneration and/or restoration of the entire nerve 115c. Regeneration of the entire nerve 115c may, for example, induce continued re-enervation (e.g., small fiber neurons) of the peripheral body portion 105. Regeneration of the vasculature 110c and/or the entire nerve 115c may, for example, cause ectodermal regeneration. Ectodermal regeneration may, for example, include improved skin health. Ectodermal regeneration may, for example, include follicle formation and growth. Follicle generation may, for example, cause restoration of hair growth. Ectodermal regeneration may, for example, include nail growth. Accordingly, the continued physical contact with the TESC 120 may, for example, advantageously slow, arrest, and/or reverse disease processes associated with the pathology.

FIG. 2A depicts an exemplary chemical structure 205 of minoxidil (National Center for Biotechnology Information. “PubChem Compound Summary for CID 4201, Minoxidil” PubChem, https://pubchem.ncbi.nlm.nih.gov/compound/Minoxidil.). FIG. In some embodiments, the VGIA 120a may, for example, include a vasodilating agent. The vasodilating agent may include, for example, a nitric oxide precursor. In some implementations, for example, the VGIA 120a may include minoxidil. Without being bound by a particular theory, minoxidil and/or nitric oxide may, for example, induce vasodilation.

Minoxidil and/or nitric oxide may, by way of example and not limitation, enhance, promote, and/or otherwise have a role in neural transmission. For example, minoxidil may act as a neuroactivator for the peripheral nervous system. Minoxidil may, by way of example and not limitation, act as a neuroactivator for the central nervous system.

Minoxidil and/or nitric oxide may, for example, promote and/or induce muscle relaxation.

In some examples, minoxidil may induce a direct response on dermal (e.g., skin) cells. For example, minoxidil may act to enhance a barrier function of dermal cells. Minoxidil may, for example, improve blood flow rate. In some examples, minoxidil may accelerate healing (e.g., of ectodermal tissue) and/or prevent dermal breakdown.

The minoxidil may, for example, be in solution. The solution may, for example, include water. The solution may, for example, include a delivery mechanism. The delivery mechanism may, by way of example and not limitation, include propylene glycol. The delivery mechanism may, for example, be selected to reduce or eliminate irritation of the skin. For example, the delivery mechanism may include glycerin. In some examples, the delivery mechanism may include PEG 400. In some implementations, by way of example and not limitation, the minoxidil may be a microemulsion in the delivery mechanism.

FIG. 2B depicts an exemplary chemical structure 210 of lactic acid (National Center for Biotechnology Information. “PubChem Compound Summary for CID 612, Lactic acid” PubChem, https://pubchem.ncbi.nlm.nih.gov/compound/612 #section=2D-Structure.). In some embodiments, the moisturizing agent 120b may, for example, include a moisturizing agent effective for moisturizing skin on peripheral body portions (e.g., limbs, hands, feet). By way of example and not limitation, the moisturizing agent 120b may, for example, include lactic acid. For example, the moisturizing agent 120b may include ammonium lactate. The moisturizing agent 120b may, for example, include an ammonium lactate cream. As an illustrative example, the ammonium lactate cream may include Lac-Hydrin® (Westwood-Squibb Pharmaceuticals Inc., Princeton, NJ).

In some implementations, the moisturizing agent 120b may be implemented as a carrier (e.g., delivery mechanism) for the VGIA 120a.

In some implementations, the moisturizing agent 120b may include an emollient. The emollient may, for example, include lanolin. The emollient may, for example, include mineral oil. In some implementations, for example, the moisturizing agent 120b may include a lanolin-mineral oil lotion (e.g., Eucerin® cream, available from Beiersdorf Inc, Stamford, CT).

FIG. 3 is a flowchart illustrating an exemplary topical ectodermal stimulation method. As depicted, a method 300 includes identifying, in a step 305, present and/or predicted future damage to peripheral vasculature. The damage may, for example, include neuropathies. Neuropathies may, by way of example and not limitation, include diabetic neuropathy. The damage may, for example, include trauma-induced devascularization. The trauma-induced pathology may, for example, include pressure damage (e.g., bed sores). For example, the method 300 may be begun to prevent bed sores (e.g., in response to a patient being bed-ridden for extended periods of time). The trauma-induced pathology may, for example, include wounds.

In a step 310, a therapeutic dose of the TESC 120 may be applied in substantially continuous topical contact with a body portion (e.g., the peripheral body portion 105). As depicted, the TESC 120 may include minoxidil and a moisturizing agent. The moisturizing agent may, for example, include ammonium lactate. If it is determined, in a decision point 315, that sufficient ectodermal stimulation has been induced, then the method 300 proceeds to a step 320. Otherwise, application of the (initial) therapeutic dose is continued.

In some implementations, by way of example and not limitation, the therapeutic dose may be substantially 1 mL of a 1%-10% minoxidil solution. In some implementations, by way of example and not limitation, the therapeutic does may, for example, be substantially 1 mL of a solution between 2%-5% minoxidil. The therapeutic dose may, for example, be applied every 12 hours (e.g., ever morning and every evening). In some implementations, by way of example and not limitation, the therapeutic dose may not exceed 2 mL in 24 hours. In some implementations, for example, a lower amount of minoxidil may be applied (e.g., <1 mL, <2% minoxidil) in an ongoing contact with the skin (e.g., in a cream, in a continuous applicator). For example, a therapeutic dose may be achieved with a lower concentration of minoxidil due to synergistic effects with the moisturizing agent (e.g., ammonium lactate).

In the decision point 315, sufficient ectodermal stimulation may, for example, be determined based on symptoms (e.g., dryness, hair growth, sensory response). In some implementations, sufficient ectodermal stimulation may be determined based on temperature (e.g., an increase in surface temperature corresponding to increased vascularization). In some implementations, sufficient ectodermal stimulation may be determined based on oxygen level in the peripheral body portion 105 (e.g., using a pulse-oximeter, with increase oxygen level corresponding to increased vascularization). In some implementations, sufficient ectodermal stimulation may be determined based on sensory response (e.g., increased level of sensory feeling corresponding to increase in enervation, such as by a graded response). In some implementations, sufficient ectodermal stimulation may be automatically determined based on predetermined criterion.

In the step 320, once sufficient ectodermal stimulation has been reached (e.g., based on predetermined criterion), a maintenance dose is applied in substantially continuous topical contact. The maintenance dose may, by way of example and not limitation, be lower than the therapeutic dose. The maintenance dose may, for example, decrease a risk of side effects while continuing to promote restoration and/or regeneration. For example, the maintenance dose may be selected to be therapeutic for reducing and/or arresting devascularization and/or de-enervation.

In a decision point 325, if it is determined that symptoms increase (e.g., de-enervation, de-vascularization, loss of sensory detection, hair loss, nail loss), then the maintenance dose is increased in a step 330. Otherwise, the method 300 returns to the step 320.

FIG. 4 depicts an exemplary field-activated continuous topical ectodermal delivery device (CTEDD). An exemplary CTEDD 405 includes a multi-layer textile 400 (e.g., fabric). The multi-layer textile 400 includes chemical-containing layers 405a, 405b, . . . , 405i−1, and 405i. The chemical containing layers are separated by separation layers 410 (separation layers 410a, 410b . . . 410i). In the depicted example, the chemical containing layer 405a includes chemical 415a, chemical containing layer 405b includes chemical 415b, chemical containing layer 405i-i includes chemical 415i−1, and chemical containing layer 405i includes chemical 415i. Together the chemicals 415a . . . 415i may form a TESC 120. For example, the chemicals may include a VGIA 120a and/or moisturizing agent 120b. The chemical may, for example, include carriers/delivery structures.

Upon application of the CTEDD 405, the separation layers 410a may be stretched or otherwise deformed. The separation layers 410a may, for example, in response to deformation, allow communication (e.g., fluid communication, diffusion) between the layers 405a . . . 405i. Accordingly, the chemicals 415a . . . 415i may mix to form the TESC 120 and absorb into a peripheral body portion 105 in contact with the chemical containing layer 405i. For example, as depicted, the chemical-containing layers 405a . . . 405i may be formed as a garment (e.g., a sock). Accordingly, the chemicals 415a . . . 415i may advantageously be manufactured, stored, delivered and then activated and kept in substantially continuous topical contact with the peripheral body portion 105.

FIG. 5 depicts activation of a field-activated CTEDD. The CTEDD 500 includes a first layer 505a and a second layer 505b separated by an impermeable layer 510. Upon application of tension (T), the impermeable layer 510 ruptures, allowing communication between the first layer 505a and the second layer 505b. Accordingly, chemicals (e.g., VGIA 120a) stored in the first layer 505a and chemicals (e.g., moisturizing agent 120b) stored in the second layer 505b. Accordingly, the TESC 120 may be mixed and delivered to a patient (e.g., via a garment, via an applicator) on demand.

FIG. 6 depicts illustrative active ingredient concentration profiles in a topical ectodermal stimulation method, such as with and without a CTEDD. As depicted, periodic direct application in a quickly drying format (e.g., water-based liquid) is shown with respect to a first concentration profile 601. Concentration 605 of the VGIA 120a in the dermal layer(s) is shown relative to a minimum therapeutic level 610. As can be seen, the concentration 605 drops below the minimum therapeutic level 610 between each application.

As depicted, substantially continuous topical application (e.g., in a cream, using a CTEDD), is shown with respect to a second concentration profile 602. Concentration 615 of the VGIA 120a in the dermal layer(s) is shown relative to the minimum therapeutic level 610. As can be seen, the concentration 615 stays above the minimum therapeutic level 610 between each application (e.g., changing the CTEDD, re-applying a cream).

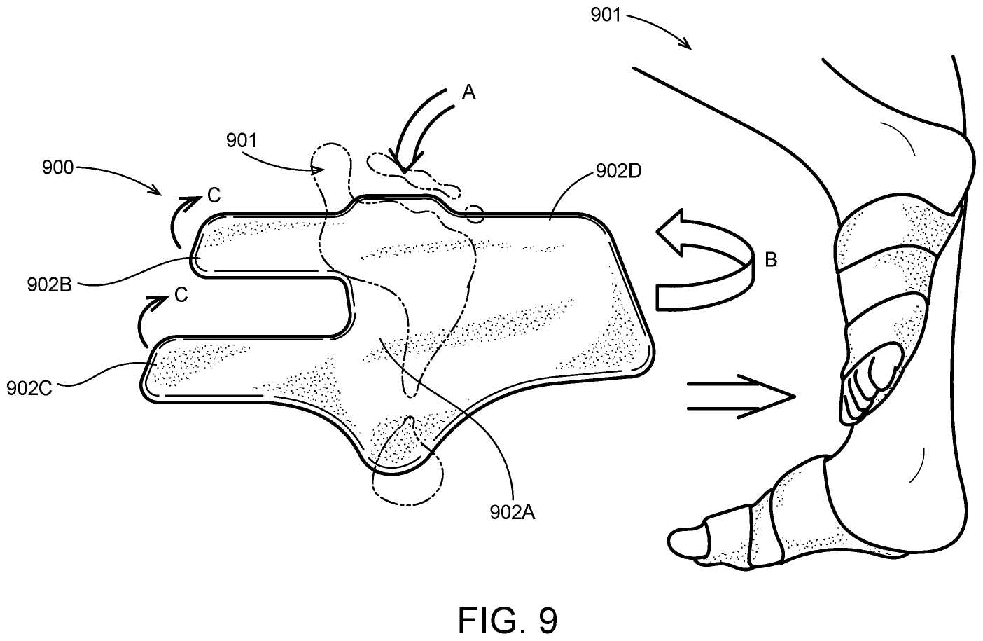

FIG. 7 depicts an illustrative kit and illustrative replaceable topical ectodermal delivery devices (RTEDDs). An illustrative topical ectodermal stimulation kit 700 includes a packaging 705. Inside the packaging is at least one TESC package 706. The least one TESC package 706 may, for example, include a quantity of the TESC 120 (e.g., as disclosed at least with reference to FIG. 1). In the depicted example, a preparatory device 710 (e.g., wipes, alcohol wipes, topical composition) is included in the packaging 705.

In some embodiments, one or more RTEDDs may be included. For example, one or more brush RTEDD 701 may be included (e.g., in the illustrative topical ectodermal stimulation kit 700, as a separate component). As depicted, the brush RTEDD 701 includes a coupler 715 configured to releasably couple in fluid communication with the TESC package 706 contents. The brush RTEDD 701 includes a lumen 720 in fluid communication with apertures opening over bristles 725. Accordingly, the contents of the TESC package 706 may be advantageously dispensed through the brush RTEDD 701 and spread using the bristles 725. As shown, the brush RTEDD 701 may be replaceable (e.g., disposable).

As an illustrative example, one or more swab RTEDD 702 may be included (e.g., in the illustrative topical ectodermal stimulation kit 700, as a separate component). As depicted, the swab RTEDD 702 includes a coupler 730 configured to releasably couple in fluid communication with the TESC package 706 contents. The swab RTEDD 702 includes a lumen 735 in fluid communication with apertures opening into a swab 740. Accordingly, the contents of the TESC package 706 may be advantageously dispensed through the swab RTEDD 702 and spread using the swab 740. As shown, the swab RTEDD 702 may be replaceable (e.g., disposable).

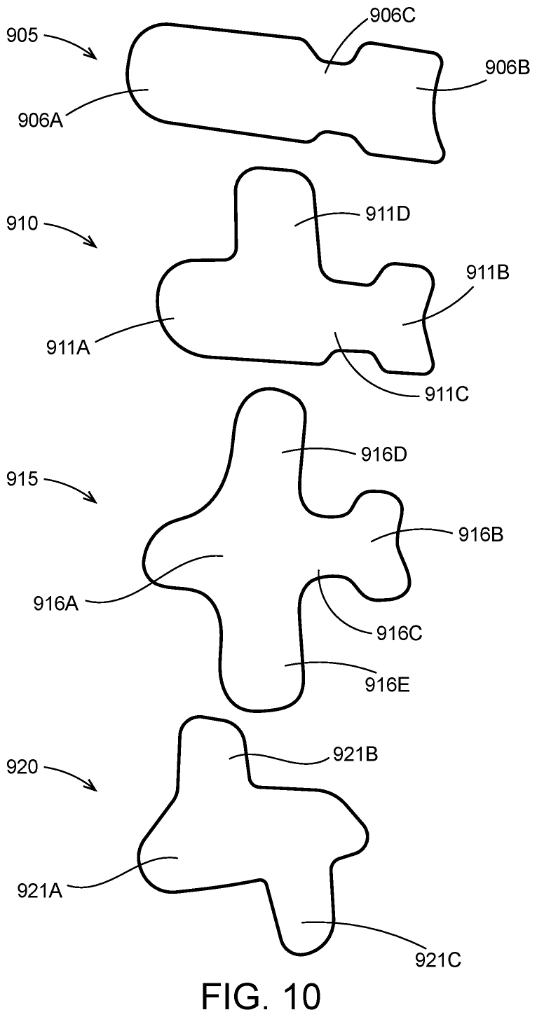

FIG. 8 depicts a block diagram of an illustrative automatic topical ectodermal delivery device (ATEDD). As depicted, a system 800 includes an ATEDD 801. The ATEDD 801 includes a controller 805 operably coupled to a sensor(s) 810. A sensor may, for example, detect time. A sensor may, for example, detect temperature. A sensor may, for example, detect oxygen level. A sensor may, for example, detect other physiological signal(s). A physiological signal may, for example, be selected to correspond to a peripheral neuropathy marker (e.g., blood flow, oxygenation level). The controller 805 is connected to a dispenser module 815. The dispenser module 815 is operably coupled (e.g., fluidly coupled) to a reservoir 820. The reservoir 820 may, for example, contain a topical stimulant (e.g., the TESC 120). The controller 805 may selectively operate the dispenser module 815 based on data received from the sensor(s) 810 to selectively dispense contents of the reservoir 820.

In the depicted example, the controller 805 is operably coupled to a datastore 806. The datastore 806 may, for example, store predetermined parameters and/or dispensing profiles associating dispensing parameters (e.g., quantities, times) with sensor inputs (e.g., current sensor inputs, historical sensor inputs). The controller 805 may, as depicted, be operably coupled to a network 825. The controller 805 may, for example, send data to and/or receive data and/or commands from remote devices via the network 825.

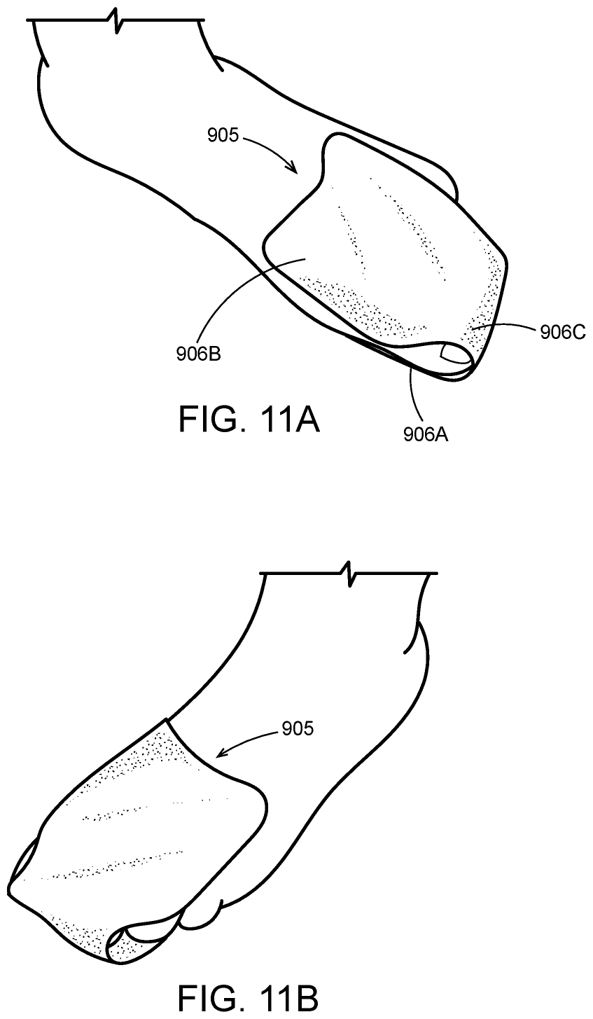

FIG. 9 depicts an exemplary adhesive CTEDD for a foot in an illustrative use-case scenario. A CTEDD 900 may, for example, include adhesive configured to be in contact with human skin. The adhesive may, for example, be configured to adhere to a foot. The adhesive layer may, for example, be impregnated with a TESC (e.g., including a VGIA). The TESC may, for example, include a moisturizing agent and/or other agent. In some implementations, the CTEDD 900 may reduce or eliminate a need for a moisturizing agent.

In the depicted example, the CTEDD 900 may be applied to a foot 901. The CTEDD 900 may be configured to wrap around and secure to the foot 901. For example, the CTEDD 900 may advantageously be easily applied to the foot 901. The CTEDD 900 may, for example, advantageously provide an easy way for a patient to substantially continuously apply the TESC to one or more target regions (e.g., with a single application of the CTEDD 900).

As depicted, the CTEDD 900 includes a main body portion defining a first surface 902A. The main body portion is connected to a second body portion (e.g., a right wing) defining a second surface 902D. The main body portion is connected to a third and fourth body portion defining a corresponding third surface 902B and a fourth surface 902C, respectively.

In an illustrative application method, as depicted, the CTEDD 900 may, for example, be prepped (e.g., removed from a sanitary packaging, removing a liner). The CTEDD 900 may, for example, be placed with an adhesive side up, as shown. The foot 901 may be placed, in a motion A, such that the bottom of the foot registers with the first surface 902A. For example, the first surface 902A may adhere to the bottom of the foot. In a motion B, the second surface 902D may be operated to register with a side (e.g., inner side) and top of the foot 901. For example, the second surface 902D may adhere to the side and/or top of the foot 901. In a motion C, the corresponding third surface 902B and/or the fourth surface 902C may be operated to register with a side (e.g., outer side) and top of the foot 901. For example, the fourth surface 902C and/or the second surface 902D may adhere to the side and/or top of the foot 901.

In some implementations, the CTEDD 900 may have one or more layers (e.g., as disclosed at least with reference to FIGS. 4-5). The layers may, for example, be continuous. The layers may, for example, be interrupted. For example, a first layer may be an outer layer. The outer layer may resist adhesive and/or active ingredients being contaminated, lost, and/or come into contact with the ambient environment.

A second layer may, for example, include adhesive. The adhesive may, for example, include hydrogel. In some implementations, the TESC may be incorporated into the adhesive layer.

Some implementations may, by way of example and not limitation, include one or more additional layers. Different layers may contain one or more active ingredients. Some layers may be blocking layers preventing fluid communication between two or more active ingredients. Blocking layers may, for example, be configured to be disrupted (e.g., tear, perforate) during use and/or application. Blocking layers may, for example, be configured to be removed prior to application.

Some implementations may, for example, include an inner layer. The inner layer may, for example, be a liner layer. The liner layer may, for example, be removable (e.g., prior to application to the foot 901).

Various implementations may advantageously achieve coverage of one or more predetermined target regions without relying on patient skill, dexterity, and/or memory. The CTEDD 900 (e.g., a bandage) may, for example, be configured to automatically register with a target region during application, such as by predetermined shape and/or alignment/fastening tabs. As an illustrative example, a heel of a foot may be a key target area for diabetic neuropathy, but may be difficult to apply TESC to and/or keep TESC continuously applied to. The CTEDD 900 may advantageously enable a patient to continuously apply a TESC to one or more predetermined target areas for at least a target duration.

In some implementations, the CTEDD 900 may be configured to elute the TESC for a predetermined time period.

In some implementations, the CTEDD 900 may, for example, include microneedles (MN). The MN may, for example, be in fluid communication with a TESC layer. The MN may advantageously increase absorption of one or more active ingredients to a target ectodermal tissue and/or layer.

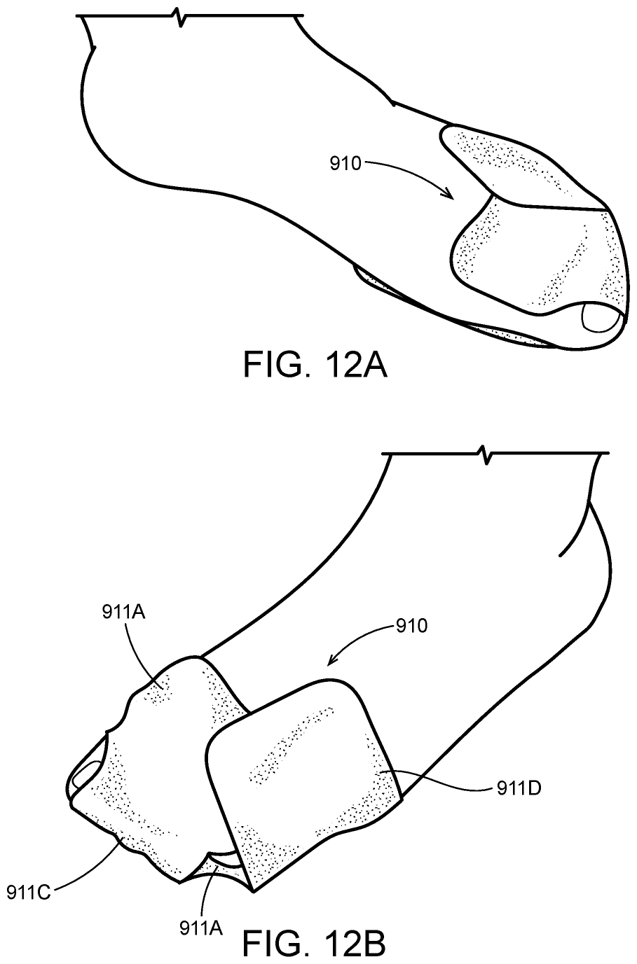

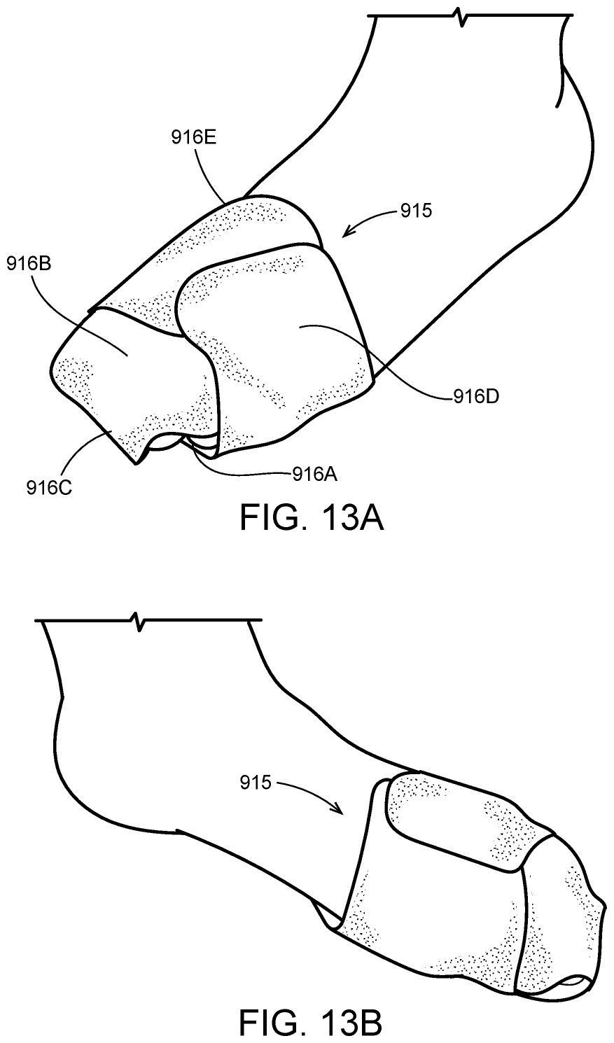

FIG. 10 depicts exemplary adhesive CTEDDs for a foot. FIG. 11, FIG. 12, FIG. 13, and FIG. 14 depict the exemplary adhesive CTEDDs of FIG. 10 in illustrative use-case scenarios.

A first CTEDD 905 is shown shaped, for example, to achieve simplified (e.g., ‘universal’) application to a bottom and top of a user's foot. The CTEDD 905, as depicted, includes a main body portion defining a first surface 906A. The main body portion is connected, via a connector segment 906C, to a second body portion defining a second surface 906B.

A second CTEDD 910 is shown configured asymmetrically, for example, to achieve application to a side portion of a user's foot. The CTEDD 910, as depicted, includes a main body portion defining a first surface 911A. The main body portion is connected, via a connector segment 911C, to a second body portion defining a second surface 911B. The main body portion is connected to a third body portion (e.g., a wing) defining a third surface 911D.

A third CTEDD 915 is shown configured with multiple wings, for example, to achieve application to multiple side portions of a user's foot. The CTEDD 915, as depicted, includes a main body portion defining a first surface 916A. The main body portion is connected, via a connector segment 916C, to a second body portion defining a second surface 916B. The main body portion is connected to a third body portion (e.g., a first wing) defining a third surface 916D. The main body portion is connected to a fourth body portion (e.g., a second wing) defining a fourth surface 916E.

A fourth CTEDD 920 may be configured to be fitted to a specific foot (e.g., left foot, right foot). The CTEDD 920, as depicted, includes a main body portion defining a first surface 921A. The main body portion is connected to a second body portion (e.g., a first wing) defining a second surface 921B. The main body portion is connected to a third body portion (e.g., a second wing) defining a third surface 921C.

In some implementations, a CTEDD may, for example, be laser cut. A CTEDD may, for example, be water-jet cut. A CTEDD may, for example, be 3D-printed. A CTEDD may, for example, be die cut. A CTEDD may, for example, be molded (e.g., cast, injection-molded, overmolded).

In some implementations, the CTEDDs 905-925 may be constructed and/or used as disclosed at least with reference to FIGS. 4-6 and 9.

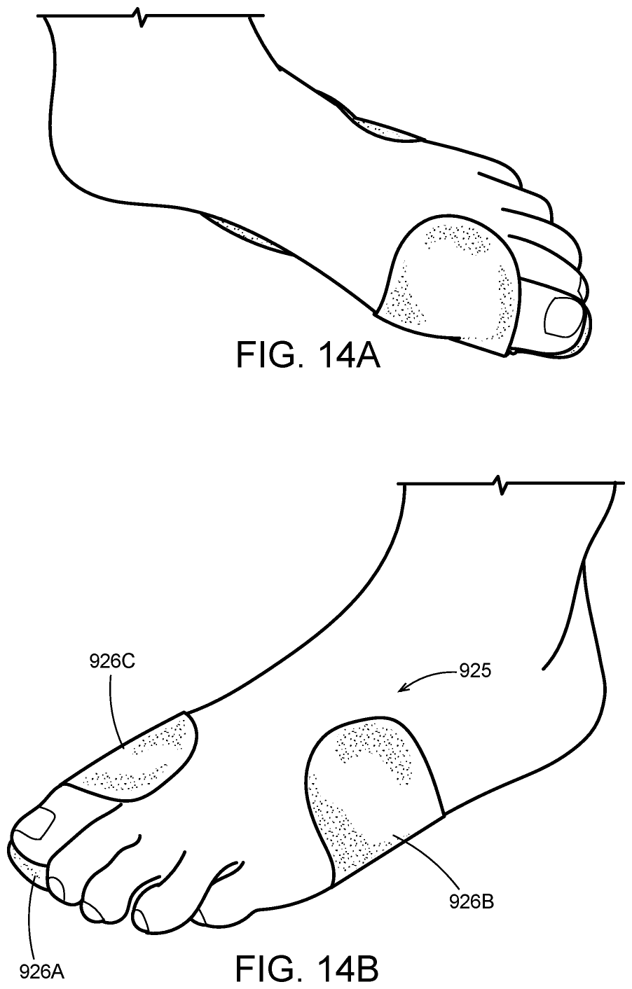

FIG. 15 depicts an illustrative portion of a CTEDD having spatially distributed TESC. A CTEDD 1500 (e.g., a bandage such as disclosed at least with reference to FIGS. 9-14) may include a base material 1505 (e.g., forming part of an application surface, such as, by way of example and not limitation, first surface 902A, corresponding third surface 902B, fourth surface 902C, and/or second surface 902D of CTEDD 900). The TESC 120 may be applied (e.g., during manufacturing, prior to application) in a spatially distributed pattern, such as shown. For example, components of the TESC 120 may be spatially separated such that the TESC 120 is only combined once a predetermined operation (e.g., application, walking after application, rubbing, removal of a liner) is performed.

As shown, the moisturizing agent 120b and the active ingredient (e.g., VGIA 120a, as depicted) are deposited in a spatial pattern of physical separation. For example, the physical separation may permit the moisturizing agent 120b to prepare the ectoderm (e.g., skin) for enhanced transport of the active ingredient. In some implementations, the physical separation may advantageously permit enhanced contact directly with the active ingredient so that active ingredient is not ‘trapped’ in the moisturizing agent 120b and/or in a base 120c without being available to the ectoderm.

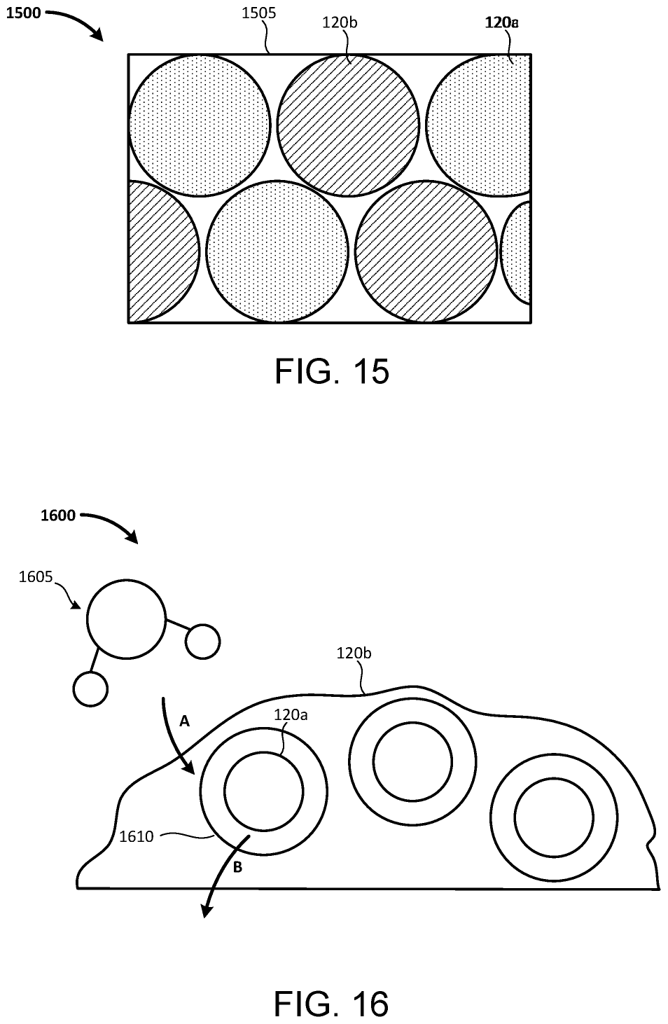

FIG. 16 depicts an illustrative TESC having active ingredient carriers configured to selectively release active ingredient. In an illustrative scenario 1600, a TESC 120 comprises a moisturizing agent 120b. The moisturizing agent 120b may include a base (e.g., base 120c). The 120a is disposed (e.g., in a microdroplet scale, in a molecular scale), as depicted, in carrier structures 1610. The carrier structures 1610 are distributed in the moisturizing agent 120b. The carrier structures 1610 may, for example, be configured for selective release in response to a carrier release operation.

For example, the carrier release operation may include exposure of the TESC 120 to moisture (e.g., to skin, to air). The carrier release operation may, for example, include application of a substance (e.g., water, solvent). For example, the carrier structures 1610 may include a microcapsule. The microcapsule may, for example, be soluble. For example, the microcapsule may be hygroscopic.

For example, the carrier release operation may include application for a predetermined period of time. For example, the carrier structures 1610 may be a time-release structure. For example, the carrier structures 1610 may dissolve over time and/or permit migration of the active ingredient out of the carrier structures 1610 over time. For example, the dissolution and/or migration may begin in response to distribution of the carrier structures 1610 in the moisturizing agent 120b.

In some implementations, for example, the carrier structures 1610 may be separated from the moisturizing agent 120b (e.g., as disclosed at least with reference to FIG. 15).

Various embodiments such as disclosed at least with reference to FIG. 16 may, for example, advantageously permit operation of the moisturizing agent 120b on an application site (e.g., to moisturize the skin) prior to application of the active ingredient. For example, the moisturizing agent 120b may precondition the ectoderm (e.g., skin). For example, the preconditioning may advantageously enhance efficacy of the active ingredient. For example, the enhanced efficacy may advantageously reduce a concentration of the active ingredient necessary to achieve and/or maintain a therapeutic effect.

Although various embodiments have been described with reference to the figures, other embodiments are possible.

Although an exemplary system has been described with reference to the figures, other implementations may be deployed in other industrial, scientific, medical, commercial, and/or residential applications.

In some implementations, the VGIA 120a may be delivered in a timed release. The timed release may, for example, be configured to address creating a concentration that penetrates thickened skin.

In some implementations, the VGIA 120a may be delivered in a foam substance. For example, the foam may advantageously carry the VGIA 120a to otherwise inaccessible areas, such as between digits (e.g., between toes), where a patient may have difficulty applying the TESC 120 otherwise.

In some implementations, the VGIA 120a may be delivered in powder form. The powder form may, for example, be activated (e.g., liquified) over time by skin moisture. The powder may, for example, ‘cling’ to the peripheral body portion 105 (e.g., the feet). Accordingly, the powder may advantageously hold the VGIA 120a in substantially continuous topical contact.

In some implementations, the VGIA 120a may be delivered using a drug eluting material. For example, the VGIA 120a and/or the moisturizing agent 120b may be impregnated into a drug eluting material. The drug eluting material may be applied to the peripheral body portion 105 (e.g., by rubbing, via a garment).

In some implementations, the TESC 120 may be delivered using a long brush with drug eluding bristles. A head of the brush, for example, may be periodically changed (e.g., daily).

In some implementations, the TESC 120 may be delivered using a stick with a drug eluding cloth. The cloth may, for example, be periodically changed (e.g., daily).

In some implementations, the VGIA 120a may be delivered from a container. A patient may, for example, engage (e.g., stand on) an actuator and the VGIA 120a may be sprayed onto the peripheral body portion 105 (e.g., their feet).

In some implementations, the VGIA 120a may be impregnated in a shoe shell. The VGIA 120a may be released into topical application with the peripheral body portion 105 (e.g., feet) when the shoe is worn.

In some implementations, the VGIA 120a may be delivered in a toe applicator (e.g., configured to encircle and/or otherwise couple to a user's toe(s)). In some implementations, the applicator may be sold as a multi-pack kit. The kit may, for example, include an accessory stick the can attach to an applicator for an easier application

In some implementations, the VGIA 120a may be delivered using a device that detects when a patient is lying down (e.g., via an accelerometer). The device may, for example, emit medication in response to lying down and/or at a predetermined time (e.g., night-time). The device may, for example, detect when the patient is sitting up (e.g., at a second predetermined time). For example, the device may emit a subsequent dose based on time, activity, and/or posture. Accordingly, the device may advantageously time delivery for substantially continuous delivery of the VGIA 120a.

In some implementations, the TESC 120 may, for example, include therapeutic cannabis-derived pharmaceuticals (CDP). For example, the TESC 120 may include CBD oil. In some implementations, the TESC 120 may include dronabinol. In some implementations, the TESC 120 may include nabilone. The CDP(s) may, for example, upregulate the CB1 receptors in the skin. Upregulation of the CB1 receptors may be neuroprotective (e.g., preventing degeneration of existing nerve cells). For example, the CDP(s) may be synergistic with the VGIA 120a (e.g., nitric oxide, nitric oxide precursor) by protecting existing nerves while inducing nerve regeneration and/or regrowth. The CB1 receptors may, for example, be highly expressed. Accordingly, CDPs may advantageously have a rapid impact on protecting existing nerve cells.

In some implementations, the TESC (e.g., the TESC 120) may, for example, include one or more moisturizing agents (e.g., natural moisturizing factor(s) (NMFs)), such as disclosed at least with reference to TABLE 1 (below). As an illustrative example, urea may be incorporated into a TESC as a moisturizing agent. Without being bound to a particular theory, urea may, by way of example and not limitation, operate to improve ectodermal permeability barrier function. The urea may, for example, enhance uptake of the VGIA (e.g., minoxidil). For example, urea may be used in place of another moisturizing agent (e.g., lactic acid). In some implementations, by way of example and not limitation, urea may be used in combination with another moisturizing agent(s). For example, multiple moisturizing agents and/or other active ingredients may be used to promote one or more target functions of the ectoderm. Urea may, for example, exhibit antimicrobial properties.

TABLE 1

| Active Ingredient | Exemplary moisturizing action |
|---|---|
| Glycerin | Humectant |
| Urea | Humectant |
| Ammonium lactate | Humectant |
| Gelatin | Humectant |
| Hyaluronic acid | Humectant |
| Lanolin | Occlusive |
| Paraffin | Occlusive |
| Petrolatum | Occlusive |
| Cholesterol | Occlusive |
| Stearyl alcohol | Occlusive |
| Petrolatum | Emollient |
| Vegetable oil | Emollient |
| Dimethicone | Emollient |
| Propylene glycol | Emollient |
| Castor oil | Emollient |
| Collagen | Rejuvenator |
| Keratin | Rejuvenator |
| Elastin | Rejuvenator |

As an illustrative example, a TESC may incorporate a urea-inclusive cream including minoxidil. The cream may, for example, include one or more NMFs other than urea. NMFs may, for example, include lactate. NMFS may, for example, include Pyrrolidinecarboxylic Acid (PCA). NMFs may, for example, include Amino Acids. NMFS may, for example, include salts. NMFs may, for example, include Glucose.

As an illustrative example, a TESC may include urea and minoxidil. The minoxidil may, for example, be present in therapeutic amounts for ectodermal stimulation. For example, urea may advantageously facilitate transport of molecules (e.g., antifungals, corticosteroids, hormones) through ectodermal tissue (e.g., skin, nails). The urea may, for example, be provided in the TESC in an effective concentration to increase transport of the minoxidil. For example, a combination of urea and minoxidil may advantageously reduce an effective concentration of minoxidil (e.g., by increasing transport of minoxidil into and/or through ectodermal tissue).

The urea may, for example, be present in therapeutic amounts for skin moisturizing and/or skin barrier function improvement. For example, urea may advantageously induce increased water absorption by keratinocytes in the presence of urea.

A TESC including a combination of urea and minoxidil may, for example, provide complimentary and/or synergistic effects. For example, urea may advantageously improve skin barrier function. For example, urea may improve antimicrobial defense. For example, urea may regulate gene expression in keratinocytes relevant for differentiation and/or antimicrobial peptide production. Urea may, for example, advantageously regulate keratinocyte proliferation (e.g., keratolytic.

Urea may, for example, be used in a TESC at a therapeutic concentration for improving skin barrier function. Urea may, for example, be used in a TESC at a therapeutic concentration for improving skin moisture. Urea may, for example, be used in a TESC at a therapeutic concentration as a keratolytic agent.

In some implementations, a TESC may include, for example, minoxidil, ammonium lactate, and urea may be provided. By way of example and not limitation, the TESC may include an effective concentration of minoxidil to induce vascular stimulation. The TESC may, for example, include an effective concentration of urea and ammonium lactate to improve transport of the minoxidil, moisturization of the skin, keratolysis, and/or improved barrier function of the ectoderm.

The TESC may, for example, include ceramides. Ceramides may, for example, operate to prevent moisture loss (e.g., due to evaporation) and/or strengthen the skin barrier. For example, ceramides may augment natural ceramides in the ectoderm (e.g., the skin barrier).

In an illustrative test with repetitive topical application of minoxidil as a VGIA 120a in patients suffering hair loss and numbness due to diabetic neuropathy, hair growth, nail growth, and temperature perception (restoration of innervation) was achieved on the leg within 2-3 weeks. The minoxidil was applied twice a day in a quantity of approximately 1 mL per day in a concentration of 2-5% minoxidil.

In some embodiments, a TESC may, for example, include ammonium lactate (AL) with a VGIA (e.g., minoxidil). AL may, by way of example and not limitation, be particularly effective in combination with a VGIA. Without being bound to a particular theory, damage in diabetic neuropathy may, for example, extend beyond the level of the nerve. For example, the nervous system may be responsible for maintaining homeostasis in the periphery including, by way of example and not limitation, skin cell health, skin hydration, and/or vascular health. Ammonium lactate (AL) may, for example, advantageously act on the skin in a number of capacities. For example, AL may advantageously act as a modulator of skin keratinization. For example, AL as a modulator of skin keratinization may be significantly more effective than lactic acid lotions (e.g., which may also be a-hydroxy acids). Ammonium lactate may, for example, produces an epidermal proliferation which may, for example, create a stimulatory response, which may, for example, result in improved skin hydration.

As an illustrative example, a TESC 120 may include a combination of minoxidil (e.g., VGIA 120a) and ammonium lactate (e.g., moisturizing agent 120b). The minoxidil may, for example, be a minoxidil powder. The TESC may, for example, include a concentration of minoxidil not exceeding a safe therapeutic level and/or a maximum concentration without precipitating out. For example, a maximum safe therapeutic level may be less than about 25%. The level may, for example, be less than about 10%. The level may, for example, be less than or equal to about 8%.

The maximum soluble concentration may, for example, be at room temperature (e.g., about seventy degrees Fahrenheit and/or twenty degrees Celsius). The maximum soluble concentration may, for example, be in an aqueous solution. The maximum soluble concentration may, for example, be less than about 25%. The maximum soluble concentration may, for example, be less than 10%. The maximum soluble concentration may, for example, be less than or equal to about 8%. The maximum soluble concentration may, for example, be less than or equal to about 5%. For example, the TESC may include about 5% minoxidil by total mass.

In some implementations, for example, minoxidil may cause headaches and/or other side effects above about 8%. The minoxidil may, for example, reduce or lose efficacy below about 5%. The ammonium lactate may, for example, be 70% ammonium lactate solution. The ammonium lactate may, for example, be included in the TESC at a sufficient concentration for moisturizing effect when non-invasively (e.g., topically) applied to a region of ectoderm (e.g., skin surface). The sufficient concentration may, for example, be less than about 15%. The sufficient concentration may, for example, be above about 10%. For example, the sufficient concentration may be between about 12% to 14%. The TESC may, for example, include 18.2% of 70% ammonium lactate by total mass. For example, the TESC may include about 12.5% of ammonium lactate by total mass.

In some implementations, ammonium lactate may induce ectoderm degradation (e.g., corrosiveness, dehydration, photocarcinegicity) above about 14%. In some implementations, ammonium lactate may reduce or lose moisturizing efficacy below about 12%.

The TESC may, for example, include propylene glycol. The glycol may, for example, be lyophilic. For example, the glycol may increase solubility of minxodil. For example, the glycol may advantageously reduce precipitation (e.g., of minoxidil). The glycol may, for example, advantageously enhance penetration (e.g., reduce penetration time) of the active ingredient (e.g., minoxidil). The glycol may, for example, increase absorption of the ectoderm. For example, the TESC may include 66.67% propylene glycol by total mass.

The TESC may, for example, include an alcohol (e.g., ethyl alcohol). The alcohol may, for example, be 100% ethyl alcohol solution. The TESC may, for example, include 10% alcohol by total mass. In some implementations, by way of example and not limitation, the alcohol may create a cooling effect on the skin. For example, the cooling effect may promote patient comfort. The alcohol may, for example, reduce an oily feel of the TESC. For example, the alcohol may promote evaporation. The alcohol may, for example, promote opening of pores in the ectoderm. For example, the alcohol may advantageously promote (e.g., increase) absorption of the TESC and/or some component(s) thereof.

In some implementations, a TESC may include sodium hyaluronic acid (SHA). The SHA may, for example, increase moisturization of the ectoderm. The SHA may, for example, increase absorption of an active ingredient (e.g., minoxidil). The SHA may, for example, increase bioavailability of the active ingredient. For example, a TESC in a cream form may, for example, include SHA (e.g., in addition to or in place of glycol).

In some implementations, a TESC may re-applied once a day. In some implementations, a TESC may, for example, be applied twice a day.

For example, a concentration of minoxidil (volume distribution, Vd) of about 3 Liters/kilogram in blood plasma may be therapeutic. For example, a dose of between 12-14% of ammonium lactate in the presence of the skin may be moisturizing. The TESC may be applied to maintain target concentrations. For example, the TESC may be reapplied no more than every 2 hours. The TESC may, for example, be reapplied no more than every 6 hours. For example, the TESC may be reapplied no more than every 10 hours. For example, the TESC may be reapplied no more than every 24 hours.

In an illustrative preparation method, alcohol may be mixed with propylene glycol. The mixture may, for example, be heated (e.g., to between 55-65° C.). The alcohol may, for example, be approximately 45% of the final volume. The container may, for example, be covered to prevent evaporation (e.g., covered with lab film, such as PARAFILM M (available from SIGMA-ALDRICH, Burlington, Massachusetts, USA). Minoxidil may, for example, be added to the mixture, while stirring and mix until dissolved. Alcohol may, for example, be added to bring the mixture to a final volume. In some implementations, by way of example and not limitation, the mixture may be disposed in an applicator (e.g., an aerosol applicator, such as a spray bottle).

Some embodiments may, for example, include a collagen stimulating agent (CSA). For example, a moisturizing agent may be selected as a CSA. In some implementations, a TESC may include at least one CSA and at least one moisturizing agent. A VGIA (e.g., minoxidil) may, for example, result in the loss of synthesis of collagen in the fibroblast. Collagen is a natural fibrous protein in the body that makes up at least part of connective tissues. Collagen may, for example, be integral to skin health. For example, collagen may be necessary in at least some situations for the prevention and/or healing of wounds. Accordingly, as an illustrative example, a combination of minoxidil and a CSA may advantageously enhance a treatment (e.g., including neuropathy) by counteracting negative impacts of minoxidil (e.g., collagen suppression) which may otherwise negatively impact skin.

In an illustrative example, a cream form of a TESC may, for example, include about 1% minoxidil powder by total mass. The TESC may, for example, include about 18.2% of 70% ammonium lactate solution, by total mass. The TESC may, for example, include about 25% of versabase gel by total mass. The TESC may, for example, include about 55.8% of emollient cream base by total mass. The TESC may, for example, be disposed in a dispenser (e.g., container, pump).

In an illustrative method, the cream form of TESC may be created by weighting minoxidil and dissolving with the ammonium lactate solution. The versabase gel may be measured and added to the minoxidil-ammonium lactate solution, and mixed well. The emollient may be added and mixed thoroughly.

“About” may, for example, refer to a measurement within customary tolerances for an appropriate method, composition, and/or apparatus. If no customary tolerances are known, about may, for example, be within +/−1. About 50% may, for example, encompass above 49% and less than 51%.

Ammonium lactate may, by way of example and not limitation, enhance collagen production and/or migration in endothelial cells. Accordingly, in some implementations, a TESC may include AL as a CSA.

Urea may, for example, enhances the synthesis of collagen. In some implementations, a TESC may, for example, include urea as a CSA.

Glycerin may, for example, induce collagen stimulation. Hyaluronic acid may, for example, induce collagen stimulation. Aloe may, for example, induce collagen stimulation. Lanolin may, for example, induce collagen stimulation. One or more collagen-stimulation induction agents may, for example, be included in a TESC (e.g., at least as a CSA).

In some implementations, a TESC may, for example, include collagen and/or collagen-like products. For example, a TESC may include collagen as a collagen auxiliary agent (CAA) (e.g., with a VGIA such as minoxidil) to supplement a loss of collagen synthesis caused by the VGIA. For example, in some implementations, gelatin may be used as a skin supplement (e.g., as a CAA) in a TESC. Accordingly, in some implementations, a TESC including a combination of minoxidil with one or several of moisturizing agents, CAA, and/or CSA may, for example, advantageously provide a symbiotic effect(s).

In some implementations, the TESC may be manufactured and/or applied as a treatment, by way of example and not limitation, for radiation induced skin injury (RSI). For example, acute RSI may involve dry and/or wet desquamation. Acute RSI may, for example, include skin necrosis. Acute RSI may, for example, include ulcers. Acute RSI may, for example, include bleeding. Chronic RSI may, for example, include chronic ulcers. Chronic RSI may, for example, include radiation-induced keratosis. A TESC may, for example, be applied at therapeutic concentrations and/or durations to induce regeneration of the injured skin. In some examples, a TESC containing minoxidil and ammonium lactate may advantageously treat acute and/or chronic RSI. In some examples, a TESC containing minoxidil and urea may, for example, advantageously treat acute and/or chronic RSI.

In some implementations, the TESC may be manufactured and/or applied as a treatment, by way of example and not limitation, for chemotherapy induced neuropathy (CIN). A TESC may, for example, be applied at therapeutic concentrations and/or durations to induce re-enervation. In some examples, a TESC containing minoxidil and ammonium lactate may advantageously treat CIN. In some examples, a TESC containing minoxidil and urea may, for example, advantageously treat CIN.

In some implementations, the TESC may be manufactured and/or applied as a treatment, by way of example and not limitation, for wound healing. A TESC may, for example, be applied at therapeutic concentrations and/or durations to induce ectodermal stimulation (e.g., nerve regeneration, vascular regeneration) in and/or around a wound site. In some examples, a TESC containing minoxidil and ammonium lactate may advantageously treat wound sites. In some examples, a TESC containing minoxidil and urea may, for example, advantageously treat wound sites.

In some implementations, the TESC may be manufactured and/or applied, by way of example and not limitation, as a post-herpetic neuralgia (PHN) treatment. A TESC may, for example, be applied at therapeutic concentrations and/or durations to induce re-enervation. In some examples, a TESC containing minoxidil and ammonium lactate may advantageously treat PHN. In some examples, a TESC containing minoxidil and urea may, for example, advantageously treat PHN.

In some implementations, the TESC may be manufactured and/or applied, by way of example and not limitation, as a prevention and/or treatment of decubitus ulcer (DU) formation. A TESC may, for example, be applied at therapeutic concentrations and/or durations to induce healing. In some examples, a TESC containing minoxidil and ammonium lactate may advantageously treat DUs. In some examples, a TESC containing minoxidil and urea may, for example, advantageously treat DUs.

In some implementations, a TESC including minoxidil and a moisturizing agent, CAA, and/or CSA (e.g., urea, ammonium lactate, collagen) may advantageously prevent and/or treat injury and/or disease from one or more external issues. For example, a patch delivering (e.g., topically) a TESC may advantageously address external and internal issues. As an illustrative example, the patch may, for example, help reduce and/or mitigate pressure, friction, shear force, and/or injurious moisture levels. As an illustrative example, the TESC may, for example, advantageously augment, regulate, and/or stimulate mechanisms responsible for regulation of local blood flow. For example, irregular local blood flow may induce formation of ulcers (e.g., DUs). The TESC may, for example, advantageously prevent DU formation and/or other disorders (e.g., such as disclosed herein).

As an illustrative example, prolonged pressure on tissues may, for example, cause capillary bed occlusion. Capillary bed occlusion may, for example, cause low oxygen levels in the affected area. Over time, the ischemic tissue may, for example, begin to accumulate toxic metabolites. Subsequently, tissue ulceration and necrosis may, for example, occur.

Patches may, for example, be configured to place prophylactically over areas that injury (e.g., ulcers) most commonly form. Such areas may, for example, include the ischium. Such areas may, for example, include the greater trochanter. Such areas may, for example, include the sacrum. Such areas may, for example, include the heel. Such areas may, for example, include the malleolus (e.g., lateral than medial). Such areas may, for example, include the occiput.

In some aspects, the techniques described herein relate to a topical ectodermal stimulation compound for use in treatment of peripheral neuropathy, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a topical ectodermal stimulation compound for use in treatment of trauma-induced ectodermal devascularization, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a topical ectodermal stimulation compound for use as a medicament, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to the use of a topical ectodermal stimulation compound for manufacture of a medicament for treatment of peripheral neuropathy, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to the use of a topical ectodermal stimulation compound for manufacture of a medicament for treatment of trauma-induced ectodermal devascularization, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to the use of a topical ectodermal stimulation compound for manufacture of a medicament for treatment of ectodermal tissue in need of stimulation, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a method of treatment or prevention of peripheral neuropathy, the method including administering a topical ectodermal stimulation compound to a patient in need of topical ectodermal stimulation, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a method of treatment or prevention of trauma-induced ectodermal devascularization, the method including administering a topical ectodermal stimulation compound to a patient in need of topical ectodermal stimulation, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a method of treatment or prevention of ectodermal degeneration, the method including administering a topical ectodermal stimulation compound to a patient in need of topical ectodermal stimulation, the compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a topical ectodermal stimulation compound including: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

In some aspects, the techniques described herein relate to a compound, wherein the first concentration of minoxidil does not exceed a maximum soluble concentration without precipitation at room temperature.

In some aspects, the techniques described herein relate to a compound, wherein room temperature is about twenty degrees Celsius.

In some aspects, the techniques described herein relate to a compound, wherein the compound is an aqueous solution and the maximum soluble concentration without precipitation at room temperature is about five percent by total mass of the compound.

In some aspects, the techniques described herein relate to a compound, wherein the first concentration of minoxidil does not exceed a therapeutically safe dose.

In some aspects, the techniques described herein relate to a compound, wherein the therapeutically safe dose is up to about eight percent.

In some aspects, the techniques described herein relate to a compound, wherein the first concentration of minoxidil is at least about two percent by total mass of the compound.

In some aspects, the techniques described herein relate to a compound, wherein the first concentration of minoxidil is at least about five percent by total mass of the compound.

In some aspects, the techniques described herein relate to a compound, wherein the first concentration of minoxidil is no more than about eight percent by total mass of the compound.

In some aspects, the techniques described herein relate to a compound, wherein the first concentration of minoxidil is no more than about five percent by total mass of the compound.

In some aspects, the techniques described herein relate to a compound, wherein the sufficient concentration to moisturize the region of the ectoderm is selected such that, upon the non-invasive application, an absorbed concentration of the active ingredient effective to stimulate peripheral nervous tissue regeneration is achieved.

In some aspects, the techniques described herein relate to a compound, wherein the absorbed concentration is maintained for at least two hours.

In some aspects, the techniques described herein relate to a compound, wherein the absorbed concentration is maintained for at least about ten hours.

In some aspects, the techniques described herein relate to a compound, wherein the sufficient concentration is at least about ten percent by total mass of the compound.

In some aspects, the techniques described herein relate to a compound, wherein the sufficient concentration is at least about twelve percent by total mass of the compound.

In some aspects, the techniques described herein relate to a compound, wherein the sufficient concentration is at least about ten percent by total mass of the compound.

In some aspects, the techniques described herein relate to a compound, wherein the sufficient concentration is between about twelve percent to fourteen percent by total mass of the compound.

In some aspects, the techniques described herein relate to a compound, wherein the sufficient concentration is at least about ten percent by total mass of the compound.

In some aspects, the techniques described herein relate to a compound, further including at least one alcohol.

In some aspects, the techniques described herein relate to a compound, further including at least one form of glycol.

In some aspects, the techniques described herein relate to a compound, wherein the at least one form of glycol includes propylene glycol.

In some aspects, the techniques described herein relate to a compound, wherein the compound is configured to be applied in aerosol form.

In some aspects, the techniques described herein relate to a compound, wherein the aerosol form is non-foaming.

In some aspects, the techniques described herein relate to a compound, wherein the alcohol is compounded such that a cooling effect on a skin of a user upon application is provided.

In some aspects, the techniques described herein relate to a compound, wherein the at least one form of glycol is compounded such that a penetration rate of the active ingredients into the region of ectoderm is increased.

In some aspects, the techniques described herein relate to a compound, wherein the region of the ectoderm includes a skin surface of a foot of a user.

In some aspects, the techniques described herein relate to a kit including: the topical ectodermal stimulation compound 1-35; and, a topical applicator.

In some aspects, the techniques described herein relate to a kit, including a dispensable container having the topical ectodermal stimulation compound disposed therein, wherein the topical applicator is configured to be coupled to the dispensable container.

In some aspects, the techniques described herein relate to a kit, wherein the topical applicator includes: at least one dispensing aperture, and at least one lumen in fluid communication with the at least one dispensing aperture and further configured to be operated into fluid communication with a container including the topical ectodermal stimulation compound, such that the topical ectodermal stimulation compound may be selectively dispensed from the container through the at least one lumen and out the at least one dispensing aperture.

In some aspects, the techniques described herein relate to a kit, wherein the topical applicator includes a brush.

In some aspects, the techniques described herein relate to a kit, wherein at least one dispensing aperture of the brush is configured to dispense the topical ectodermal stimulation compound out during a brushing motion for distribution by bristles of the brush.

In some aspects, the techniques described herein relate to a kit, wherein the topical applicator includes a swab.

In some aspects, the techniques described herein relate to a kit, wherein at least one dispensing aperture of the swab is configured to dispense the topical ectodermal stimulation compound out during a swabbing motion for distribution by the swab.

In some aspects, the techniques described herein relate to a bandage including: a first surface configured to register with and releasably couple to cover at least about fifty percent of a surface area of a bottom of a foot of a patient; at least one second surface connected to the first surface and configured to register with and releasably couple to a top of the foot; and, a topical ectodermal stimulation compound including an active ingredient and a moisturizing ingredient, wherein the first surface and the at least one second surface are configured such that, after registration with the foot, the active ingredient and the moisturizing ingredient are in contact with the foot.

In some aspects, the techniques described herein relate to a bandage, wherein at least one of the first surface and the at least one second surface include an adhesive configured to adhere to the foot.

In some aspects, the techniques described herein relate to a bandage, wherein the at least one second surface is configured to cover at least about fifty percent of a surface area of the top of the foot.

In some aspects, the techniques described herein relate to a bandage, wherein the at least one second surface includes a first wing and a second wing.

In some aspects, the techniques described herein relate to a bandage, wherein the first wing and the second wing are each connected to a same edge of the first surface.

In some aspects, the techniques described herein relate to a bandage, wherein the first wing extends from an opposite edge of the first surface from the second wing.

In some aspects, the techniques described herein relate to a bandage, wherein the bandage is configured to be disposable.

In some aspects, the techniques described herein relate to a bandage, wherein the topical ectodermal stimulation compound is 1-34.

In some aspects, the techniques described herein relate to a bandage, wherein the topical ectodermal stimulation compound is pre-applied to at least one of the first surface and the at least one second surface.

In some aspects, the techniques described herein relate to a kit including the bandage, wherein the topical ectodermal stimulation compound is independently packaged with the bandage to be applied to at least one of the first surface and the at least one second surface prior to application.

In some aspects, the techniques described herein relate to a bandage, wherein at least one of the first surface and the at least one second surface are constructed from a multi-component material such that the active ingredient and the moisturizing ingredient are physically separated until after application of the bandage to the foot.

In some aspects, the techniques described herein relate to a bandage, wherein the multi-component material includes a plurality of layers, wherein the active ingredient is in a first layer and the moisturizing ingredient is in a second layer.

In some aspects, the techniques described herein relate to a bandage, wherein the second layer is disposed closest to the foot upon application.

In some aspects, the techniques described herein relate to a bandage, further including a third layer disposed between the first layer and the second layer, wherein: in a stowage state, the third layer separates the first layer from the second layer such that combination of the active ingredient with the moisturizing ingredient is prevented, and in a dispensing state, the third layer permits combination of the active ingredient with the moisturizing ingredient.

In some aspects, the techniques described herein relate to a bandage, wherein the third layer is operated into the dispensing state at least partially by rupture of the third layer.

In some aspects, the techniques described herein relate to a bandage, wherein the multi-component material includes a spatial distribution of adjacent deposits of either the active ingredient or the moisturizing ingredient such that, in a stowage state, combination of the active ingredient with the moisturizing ingredient is prevented.

In some aspects, the techniques described herein relate to a bandage, wherein the multi-component material includes a spatial distribution of cells, each containing one of the adjacent deposits.

In some aspects, the techniques described herein relate to a bandage wherein, in a dispensing state, the adjacent deposits are combined.

In some aspects, the techniques described herein relate to a compound, further including carrier structures including the active ingredient, such that the carrier structures release the active ingredient in response to a carrier release operation.

In some aspects, the techniques described herein relate to a compound, wherein the carrier release operation includes exposing the compound to at least one of environmental air and a skin surface, the carrier structures include a water-soluble and hygroscopic coating, and the carrier structures releasing the active ingredient includes the dissolving of the carrier structures.

In some aspects, the techniques described herein relate to a compound, wherein the carrier structures include a time-release granule, and the carrier release operation includes application for a predetermined period of time.

In some aspects, the techniques described herein relate to a compound, wherein the carrier structures are spatially distributed amongst the moisturizing ingredient.

In some aspects, the techniques described herein relate to a kit including the bandage, in a disposable sanitary wrapper.

In some aspects, the techniques described herein relate to a kit including the bandage.

In some aspects, the techniques described herein relate to a method 7-9, further including application 36-66.

Clause 1. A topical ectodermal stimulation compound for use in treatment of peripheral neuropathy, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 2. A topical ectodermal stimulation compound for use in treatment of trauma-induced ectodermal devascularization, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 3. A topical ectodermal stimulation compound for use as a medicament, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 4. The use of a topical ectodermal stimulation compound for manufacture of a medicament for treatment of peripheral neuropathy, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 5. The use of a topical ectodermal stimulation compound for manufacture of a medicament for treatment of trauma-induced ectodermal devascularization, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 6. The use of a topical ectodermal stimulation compound for manufacture of a medicament for treatment of ectodermal tissue in need of stimulation, the compound comprising: an active ingredient including a first concentration of minoxidil, a pharmaceutically acceptable salt, or a solvate thereof; and, a moisturizing agent including ammonium lactate, a pharmaceutically acceptable salt, or a solvate thereof in a sufficient concentration to moisturize a region of ectoderm upon non-invasive application of the compound.

Clause 7. A method of treatment or prevention of peripheral neuropathy, the method comprising administering a topical ectodermal stimulation compound to a patient in need of topical ectodermal stimulation, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 8. A method of treatment or prevention of trauma-induced ectodermal devascularization, the method comprising administering a topical ectodermal stimulation compound to a patient in need of topical ectodermal stimulation, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 9. A method of treatment or prevention of ectodermal degeneration, the method comprising administering a topical ectodermal stimulation compound to a patient in need of topical ectodermal stimulation, the compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 10. A topical ectodermal stimulation compound comprising: an active ingredient comprising a first concentration of minoxidil, a pharmaceutically acceptable salt, or a solvate thereof; and, a moisturizing agent comprising ammonium lactate, a pharmaceutically acceptable salt, or a solvate thereof in a sufficient concentration to moisturize a region of ectoderm upon non-invasive application of the compound.

Clause 11. The compound of any of clauses 1-10, wherein the first concentration of minoxidil does not exceed a maximum soluble concentration without precipitation at room temperature.

Clause 12. The compound of clause 11, wherein room temperature is about twenty degrees Celsius.

Clause 13. The compound of any of clauses 11-12, wherein the compound is an aqueous solution and the maximum soluble concentration without precipitation at room temperature is about five percent by total mass of the compound.

Clause 14. The compound of any of clauses 1-13, wherein the first concentration of minoxidil does not exceed a therapeutically safe dose.

Clause 15. The compound of clause 14, wherein the therapeutically safe dose is up to about eight percent.

Clause 16. The compound of any of clauses 1-15, wherein the first concentration of minoxidil is at least about two percent by total mass of the compound.

Clause 17. The compound of any of clauses 1-16, wherein the first concentration of minoxidil is at least about five percent by total mass of the compound.

Clause 18. The compound of any of clauses 1-17, wherein the first concentration of minoxidil is no more than about eight percent by total mass of the compound.

Clause 19. The compound of any of clauses 1-18, wherein the first concentration of minoxidil is no more than about five percent by total mass of the compound.

Clause 20. The compound of any of clauses 1-19, wherein the sufficient concentration to moisturize the region of the ectoderm is selected such that, upon the non-invasive application, an absorbed concentration of the active ingredient effective to stimulate peripheral nervous tissue regeneration is achieved.

Clause 21. The compound of clause 20, wherein the absorbed concentration is maintained for at least two hours.

Clause 22. The compound of any of clauses 20-21, wherein the absorbed concentration is maintained for at least about ten hours.

Clause 23. The compound of any of clauses 1-22, wherein the sufficient concentration is at least about ten percent by total mass of the compound.

Clause 24. The compound of any of clauses 1-23, wherein the sufficient concentration is at least about twelve percent by total mass of the compound.

Clause 25. The compound of any of clauses 1-24, wherein the sufficient concentration is at least about ten percent by total mass of the compound.

Clause 26. The compound of any of clauses 1-25, wherein the sufficient concentration is between about twelve percent to fourteen percent by total mass of the compound.

Clause 27. The compound of any of clauses 1-26, wherein the sufficient concentration is at least about ten percent by total mass of the compound.

Clause 28. The compound of any of clauses 1-27, further comprising at least one alcohol.

Clause 29. The compound of any of clauses 1-28, further comprising at least one form of glycol.

Clause 30. The compound of clause 29, wherein the at least one form of glycol comprises propylene glycol.

Clause 31. The compound of any of clauses 1-30, wherein the compound is configured to be applied in aerosol form.

Clause 32. The compound of clause 31, wherein the aerosol form is non-foaming.

Clause 33. The compound of any of clauses 28-32, wherein the alcohol is compounded such that a cooling effect on a skin of a user upon application is provided.

Clause 34. The compound of any of clauses 28-33, wherein the at least one form of glycol is compounded such that a penetration rate of the active ingredients into the region of ectoderm is increased.

Clause 35. The compound of any of clauses 1-34, wherein the region of the ectoderm comprises a skin surface of a foot of a user.

Clause 36. A kit comprising: the topical ectodermal stimulation compound according to any of clauses 1-35; and, a topical applicator.

Clause 37. The kit of clause 36, comprising a dispensable container having the topical ectodermal stimulation compound disposed therein, wherein the topical applicator is configured to be coupled to the dispensable container.

Clause 38. The kit of any of clauses 36-37, wherein the topical applicator comprises: at least one dispensing aperture, and at least one lumen in fluid communication with the at least one dispensing aperture and further configured to be operated into fluid communication with a container comprising the topical ectodermal stimulation compound, such that the topical ectodermal stimulation compound may be selectively dispensed from the container through the at least one lumen and out the at least one dispensing aperture.

Clause 39. The kit of any of clauses 36-38, wherein the topical applicator comprises a brush.

Clause 40. The kit of clause 39, wherein at least one dispensing aperture of the brush is configured to dispense the topical ectodermal stimulation compound out during a brushing motion for distribution by bristles of the brush.

Clause 41. The kit of any of clauses 36-38, wherein the topical applicator comprises a swab.

Clause 42. The kit of clause 41, wherein at least one dispensing aperture of the swab is configured to dispense the topical ectodermal stimulation compound out during a swabbing motion for distribution by the swab.

Clause 43. A bandage comprising: a first surface configured to register with and releasably couple to cover at least about fifty percent of a surface area of a bottom of a foot of a patient; at least one second surface connected to the first surface and configured to register with and releasably couple to a top of the foot; and, a topical ectodermal stimulation compound comprising an active ingredient and a moisturizing ingredient, wherein the first surface and the at least one second surface are configured such that, after registration with the foot, the active ingredient and the moisturizing ingredient are in contact with the foot.

Clause 44. The bandage of clause 43, wherein at least one of the first surface and the at least one second surface comprise an adhesive configured to adhere to the foot.

Clause 45. The bandage of any of clauses 43-44, wherein the at least one second surface is configured to cover at least about fifty percent of a surface area of the top of the foot.

Clause 46. The bandage of any of clauses 43-45, wherein the at least one second surface comprises a first wing and a second wing.

Clause 47. The bandage of clause 46, wherein the first wing and the second wing are each connected to a same edge of the first surface.

Clause 48. The bandage of clause 46, wherein the first wing extends from an opposite edge of the first surface from the second wing.

Clause 49. The bandage of any of clauses 43-48, wherein the bandage is configured to be disposable.

Clause 50. The bandage of any of clauses 43-49, wherein the topical ectodermal stimulation compound is according to the compound of any of claims 1-34.

Clause 51. The bandage of any of clauses 43-50, wherein the topical ectodermal stimulation compound is pre-applied to at least one of the first surface and the at least one second surface.

Clause 52. A kit comprising the bandage of any of clauses 43-50, wherein the topical ectodermal stimulation compound is independently packaged with the bandage to be applied to at least one of the first surface and the at least one second surface prior to application.

Clause 53. The bandage of any of clauses 43-52, wherein at least one of the first surface and the at least one second surface are constructed from a multi-component material such that the active ingredient and the moisturizing ingredient are physically separated until after application of the bandage to the foot.

Clause 54. The bandage of clause 53, wherein the multi-component material comprises a plurality of layers, wherein the active ingredient is in a first layer and the moisturizing ingredient is in a second layer.

Clause 55. The bandage of clause 54, wherein the second layer is disposed closest to the foot upon application.

Clause 56. The bandage of clause 55, further comprising a third layer disposed between the first layer and the second layer, wherein: in a stowage state, the third layer separates the first layer from the second layer such that combination of the active ingredient with the moisturizing ingredient is prevented, and in a dispensing state, the third layer permits combination of the active ingredient with the moisturizing ingredient.

Clause 57. The bandage of clause 56, wherein the third layer is operated into the dispensing state at least partially by rupture of the third layer.

Clause 58. The bandage of clause 53, wherein the multi-component material comprises a spatial distribution of adjacent deposits of either the active ingredient or the moisturizing ingredient such that, in a stowage state, combination of the active ingredient with the moisturizing ingredient is prevented.

Clause 59. The bandage of clause 58, wherein the multi-component material comprises a spatial distribution of cells, each containing one of the adjacent deposits.

Clause 60. The bandage of any of clauses 58-59 wherein, in a dispensing state, the adjacent deposits are combined.

Clause 61. The compound of any of clauses 1-60, further comprising carrier structures comprising the active ingredient, such that the carrier structures release the active ingredient in response to a carrier release operation.

Clause 62. The compound of clause 61, wherein the carrier release operation comprises exposing the compound to at least one of environmental air and a skin surface, the carrier structures comprise a water-soluble and hygroscopic coating, and the carrier structures releasing the active ingredient comprises the dissolving of the carrier structures.

Clause 63. The compound of any of clauses 61-62, wherein the carrier structures comprise a time-release granule, and the carrier release operation comprises application for a predetermined period of time.

Clause 64. The compound of any of clauses 61-63, wherein the carrier structures are spatially distributed amongst the moisturizing ingredient.

Clause 65. A kit comprising the bandage of any of clauses 43-64, in a disposable sanitary wrapper.

Clause 66. The kit of any of clauses 36-42 comprising the bandage of any of claims 43-65.

Clause 67. The method of treatment of any of clauses 7-9, further comprising application of the topical ectodermal stimulation compound according to any of claims 36-66.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1-6. (canceled)

7. The method of claim 9, wherein the ectodermal degeneration comprises peripheral neuropathy.

8. The method of claim 9, wherein the ectodermal degeneration comprises trauma-induced ectodermal devascularization.

9. A method of treatment or prevention of ectodermal degeneration, the method comprising administering a topical ectodermal stimulation compound (120) to a patient in need of topical ectodermal stimulation, the compound comprising:
an active ingredient (120a) comprising a first concentration of minoxidil, a pharmaceutically acceptable salt, or a solvate thereof; and,
a moisturizing agent (120b) comprising ammonium lactate, a pharmaceutically acceptable salt, or a solvate thereof in a sufficient concentration between ten percent and fifteen percent to moisturize a region of ectoderm upon non-invasive application of the compound.

10. (canceled)

11. The compound of claim 9, wherein the first concentration of minoxidil does not exceed a maximum soluble concentration without precipitation at room temperature.

12. The compound of claim 11, wherein room temperature is twenty degrees Celsius.

13. The compound of claim 11, wherein the compound is an aqueous solution and the maximum soluble concentration without precipitation at room temperature is five percent by total mass of the compound.

14. The compound of claim 11, wherein the first concentration of minoxidil does not exceed a therapeutically safe dose up to eight percent.

15. (canceled)

16. The compound of claim 11, wherein the first concentration of minoxidil is at least about two percent by total mass of the compound.

17. The compound of claim 11, wherein the first concentration of minoxidil is at least about five percent by total mass of the compound.

18. The compound of claim 11, wherein the first concentration of minoxidil is no more than about eight percent by total mass of the compound.

19. The compound of claim 11, wherein the first concentration of minoxidil is no more than about five percent by total mass of the compound.

20. The compound of claim 11, wherein the sufficient concentration between ten percent and fifteen percent to moisturize the region of the ectoderm is selected such that, upon the non-invasive application, an absorbed concentration of the active ingredient effective to stimulate peripheral nervous tissue regeneration is achieved.

21. The compound of claim 20, wherein the absorbed concentration is maintained for at least two hours.

22. The compound of claim 20, wherein the absorbed concentration is maintained for at least ten hours.

23. The compound of claim 11, wherein the sufficient concentration is at least ten percent by total mass of the compound.

24. The compound of claim 11, wherein the sufficient concentration is at least twelve percent by total mass of the compound.

25. The compound of claim 11, wherein the sufficient concentration is at least ten percent by total mass of the compound.

26. The compound of claim 11, wherein the sufficient concentration is between twelve percent to fourteen percent by total mass of the compound.

27. The compound of claim 11, wherein the sufficient concentration is at least ten percent by total mass of the compound.

28. The compound of claim 11, further comprising at least one alcohol.

29. The compound of claim 11, further comprising at least one form of glycol.

30. The compound of claim 29, wherein the at least one form of glycol comprises propylene glycol.

31. The compound of a claim 11, wherein the compound is configured to be applied in aerosol form.

32. The compound of claim 31, wherein the aerosol form is non-foaming.

33. The compound of claim 28, wherein the alcohol is compounded such that a cooling effect on a skin of a user upon application is provided.

34. The compound of claim 2, wherein the at least one form of glycol is compounded at ten percent by total mass such that a penetration rate of the active ingredients into the region of ectoderm is increased.

35. The compound of claim 11, wherein the region of the ectoderm comprises a skin surface of a foot of a user.

36-60. (canceled)

61. The compound of claim 11, further comprising carrier structures comprising the active ingredient, such that the carrier structures release the active ingredient in response to a carrier release operation.

62. The compound of claim 61, wherein the carrier release operation comprises exposing the compound to at least one of environmental air and a skin surface, the carrier structures comprise a water-soluble and hygroscopic coating, and the carrier structures releasing the active ingredient comprises the dissolving of the carrier structures.

63. The compound of claim 61, wherein the carrier structures comprise a time-release granule, and the carrier release operation comprises application for a predetermined period of time.

64. The compound of claim 61, wherein the carrier structures are spatially distributed amongst the moisturizing ingredient.

65-67. (canceled)
